(19) United States
(12) Patent Application Publication
(10) Pub. No.: US 2009/0081602 A1
(43) Pub. Date: Mar. 26, 2009

(54) AUTOMATIC MAXILLARY EXPANDER AND TRANSFERING APPARATUS

(76) Inventor: Mustafa Ayan, Konya (TR)

Correspondence Address: AKERMAN SENTERFITT, P.O. BOX 3188, WEST PALM BEACH, FL, 33402-3188, US

(21) Appl. No.: 12/297,609

(22) PCT Filed: Apr. 10, 2007
(86) PCT No.: PCT/TR07/00032

§ 371 Date: Oct. 17, 2008

(30) Foreign Application Priority Data
Apr. 17, 2006 (TR) 2006/01622
Oct. 30, 2006 (TR) 2006/05990

(51) Int. Cl. A61C 3/00; A61B 17/58

(52) U.S. Cl. 433/7; 606/90

(57) ABSTRACT

An automatic Maxillary Expander, which is a bone-borne distractor for expanding the maxillary bone in adult and adolescents having transversal maxillary hypoplasia. It fixes itself to the palatal vault in a way without any need for screwing, by the asymmetrical triangular prism-shaped spikes on the anterior, posterior columns. Both being hygienic and not wasting a bulky space in the mouth, it provides a high patient comfort. The maxillary expanding process does not interrupt orthodontic treatment of patients and minimizes damage to the texture of the mouth. An automatic Maxillary Expander Transferring Apparatus enables the practitioner to place the Automatic Maxillary Expander into the palatal surface with ease and precision into the palate under local anesthesia quickly, without any surgical operation. In addition, this apparatus is composed of a very simple mechanism. It has rounded ends in order not to hurt the practitioner or patient.

### BACKGROUND OF THE INVENTION

Transversal maxillary hypoplasia, of adolescents and adults, is frequently seen as an acquired or congenital deformity including cleft patients. In skeletally mature patients the Uni- or bilateral maxillary hypoplasia can be corrected by means of Surgically Assisted Rapid Maxillary Expansion (SARME). In conventional rapid maxillary expansion appliances, known as hyrax appliance, impressions are carried out in the patient and hyrax appliance is soldered on the bands of molar teeth or an acrylic capping is cemented on all the teeth. These tooth-borne appliances may cause periodontal problems, buccal root resorption, cortical fenestration, segmental tipping and anchorage-tooth tipping. Therefore, it may cause loss of anchorage. The tooth-borne appliances can lead to dental caries with mentally retarded patients and patients having poor oral hygiene. Therefore, during maxillary expansion process, tooth-borne appliances may lead to undesired consequences on the part of patient and dentist.

In contrast, bone-borne distractors are applied at a higher level in the palatal vault. It is possible to obtain mainly skeletal expansion with this type of distractors. In addition, tooth tipping does not occur as the forces are directly focused on the bone. However, there may be some undesired effects with this distractor. The now commercially available bone-borne distractors (e.g. Transpalatal Distractor and Magdenburg Palatal Distractor) have to be fixed with screws and have proven to be useful in acquired deformation patients. MDO-R and Rotterdam distractors do not require screws. However, these distractors are bulky and cause discomfort to the patient. Furthermore, in Rotterdam distractor, the amount of expansion decreases by each activation due to the design of the distractor. For this reason, the patient may not achieve adequate amount of daily expansion. J. Koudstaal et al. mentioned this issue in their preliminary study regarding this distractor.

### SUMMARY OF THE INVENTION

The present invention relates to an Automatic Maxillary Expander (7) which is a modified design of Hyrax appliance which includes titanium columns (1, 2) and asymmetrical triangular prism-shaped titanium spikes (3). The spikes (3) are the most important parts which conduct directly to the bone the effect of the expander at each turn of the geared co-axial screw (5) and which provide the stability of the distractor in the bone. It is applied to the palatal surface properly under local anesthesia conditions to the maxillary in need of expansion. The spikes (3), with their notched (3.1) specific designs, penetrate in to the bone by piercing mucosa with each turn of the geared co-axial screw (5). The columns (1, 2) attached to the expanding carrier blocks (4) with preferable angles employ expansion force to maxillary by the spikes (3). Each turn of geared co-axial screw (5), delivers the planned amount of daily expansion force to the maxillary. During expansion, the changeable angle at the joint between the columns (1, 2) and spikes (3) provides a better penetration of the spikes (3) into the bone at each maxillary movement, thus increasing the stability of the distractor. Since the distractor is not supported by teeth, no dental tipping is caused. The patient comfort is enhanced due to its significantly smaller size. Automatic Maxillary Expander (7) is a distractor which can be used especially to expand maxillary in adult and adolescents who have maxillary constriction. The nasal passage of the patients who have maxillary constriction expands by the expansion process and the expanded nasal air passage helps the patient nasal breathe normally. While it provides maxillary expansion in the middle line, it expands the diestema between the central incisors, making space for proper tooth contact to improve dental crowding. It provides a shorter orthodontic treatment period and the patients orthodontic treatments have not been interrupted during the expansion process. It also helps to shorten the chair time as much as possible and it is considered as the main advantage.

Automatic Maxillary Expander (7) consists of two carrier blocks (4), a screw housing (5.2) formed in the middle part of the carrier blocks (4), shaft housings (6.1) formed on the carrier blocks (4), a geared co-axial screw (5), which is mounted in screw housing (5.2), stability shafts (6) which are situated on both sides of the geared co-axial screw (5) in shaft housings (6.1) and spikes (3) mounted on the anterior and posterior columns (1,2) which are fixed to carrier blocks (4) for providing penetration and anti-rotation of the automatic maxillary expander (7) into the palatal bone. The anterior column (1) has an anti-rotation notch (1.1) which prevents rotation into the palatal vault, the posterior column (2) has an anti-rotation notch (2.1) which prevents rotation into the palatal vault; and the spikes (3) have notches (3.1) formed on the wider surfaces thereof facing the tongue for providing, stability and penetrating the spikes (3) deeply by cropping the palatal bone. The geared co-axial screw (5) includes two bores (5.3) formed thereon which are intersecting each other at 90 degrees in the middle. In addition, geared co-axial screw (5) includes a gear (5.1) having a certain number of teeth. Activation key is inserted in the opening of these bores (5.3) to rotate the geared co-axial screw (5). As the Automatic Maxillary Expander (7) engages the special Transferring Apparatus (22) with the help of the gear (5.1) on the geared co-axial screw (5), the practitioner can easily place it on the patient's palate.

### BRIEF DESCRIPTION OF THE DRAWINGS

Views of invention components:

FIG. 1: View of Automatic Maxillary Expander (7) from the top and side perspective

FIG. 1A: View of the demounted Automatic Maxillary Expander (7) from the top and side perspective

FIG. 1B: View of the mounted carrier block (4), anterior, posterior columns (1, 2) and asymmetrical triangular prism-shaped spikes (3) from the top and side perspective

FIG. 1C: View of Automatic Maxillary Expander (7) from the bottom and side perspective

FIG. 2: View of the mounted Automatic Maxillary Expander (7) and Transferring Apparatus (22) and detailed view of the mounted Automatic Maxillary Expander (7) and Transferring Apparatus (22)

FIG. 3A: Sectional view of the Automatic Maxillary Expander Transferring Apparatus (22)

FIG. 3B: View of Automatic Maxillary Expander Transferring Apparatus (22) from the back perspective

FIG. 3C: View of Automatic Maxillary Expander Transferring Apparatus (22) from the top perspective

FIG. 4: Sectional view of the mounted Automatic Maxillary Expander (7) and Transferring Apparatus (22).

FIG. 4A: Detailed sectional view of the mounted Automatic Maxillary Expander (7) and Transferring Apparatus (22)

FIG. 5A: Sectional view of handhold (21) from the top and side perspective

FIG. 5B: View of fulcrum (8) from the top and side perspective

FIG. 5C: View of crowbar arc (19) from the top and side perspective

FIG. 5D: View of crowbar (9) from the top and side perspective

FIG. 5E: Sectional view of creamier gear (14) from the top and side perspective

FIG. 5F: Sectional view of cradle (13) from the top and side perspective

FIG. 6A. Angular view of retentive arc (16) from the top and side perspective

FIG. 6B: Angular view of gear (15) from the top and side perspective

FIG. 6C: Angular view of crowbar pin (10) from the top and side perspective

FIG. 6D: Angular view of cradle gear pin (11) from the top and side perspective

FIG. 6E: Angular-view of arc pin (17) from the top and side perspective

FIG. 6F: Angular views of back stop (20) from the side perspective

FIG. 6G: Angular views of cradle bolts (18) from the side perspective

FIG. 6H: Angular views of Retentive arc bolt (12) from the side perspective

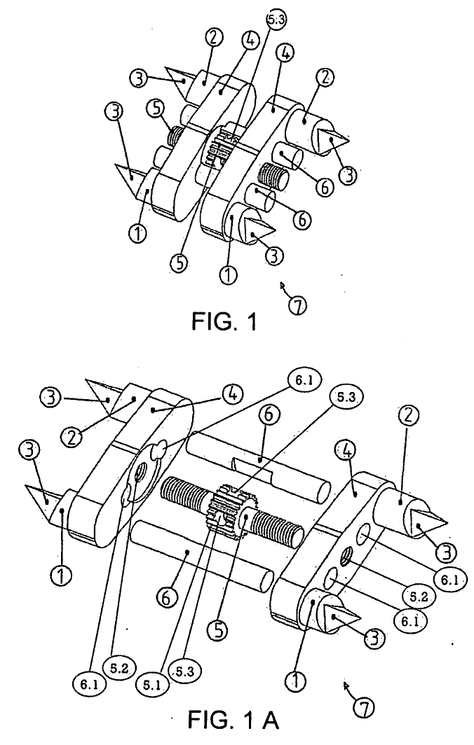

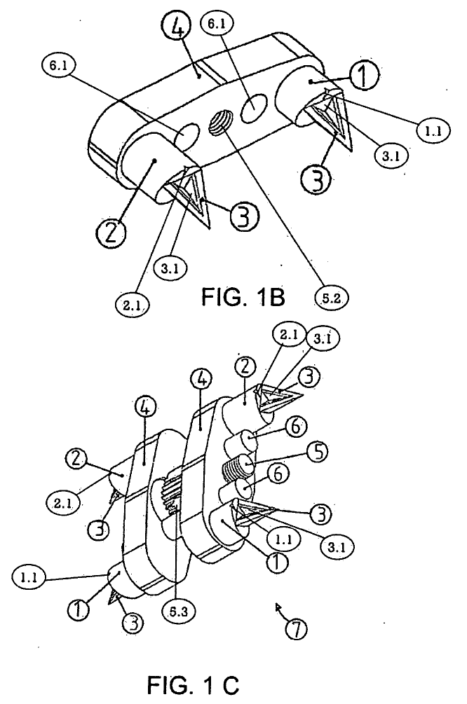

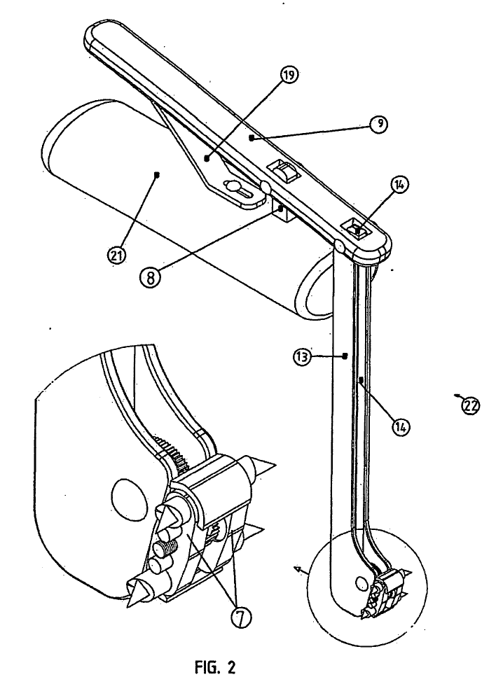

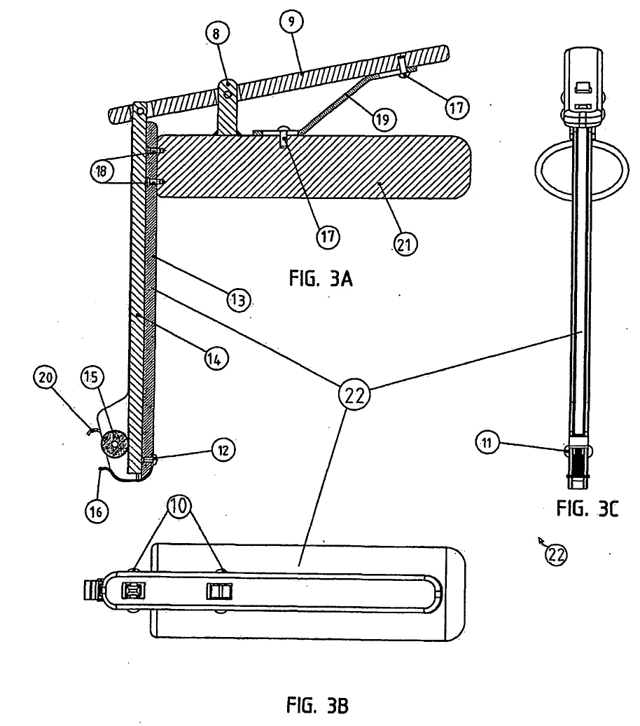

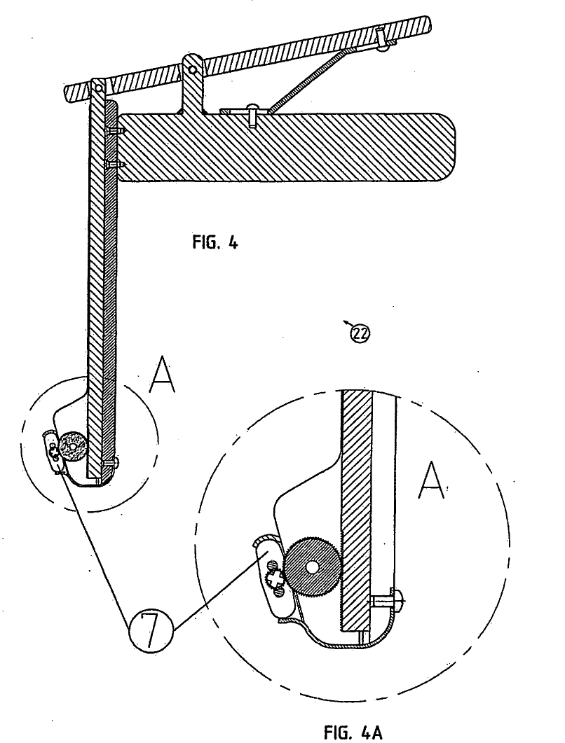

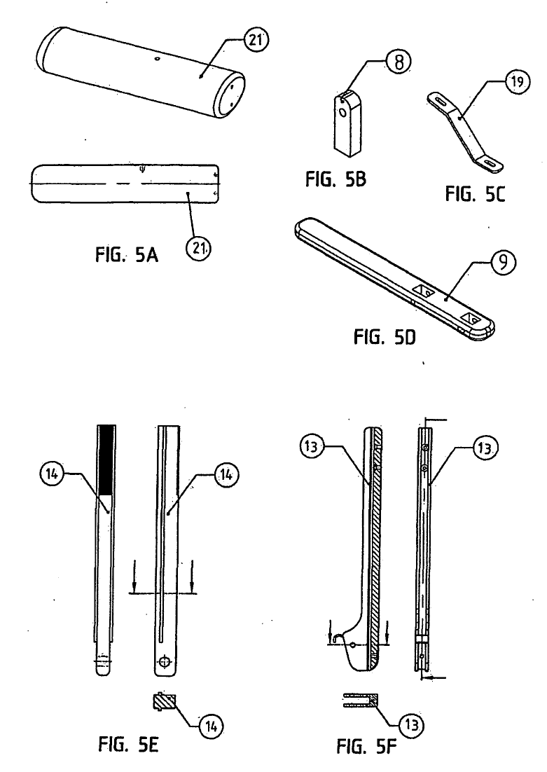

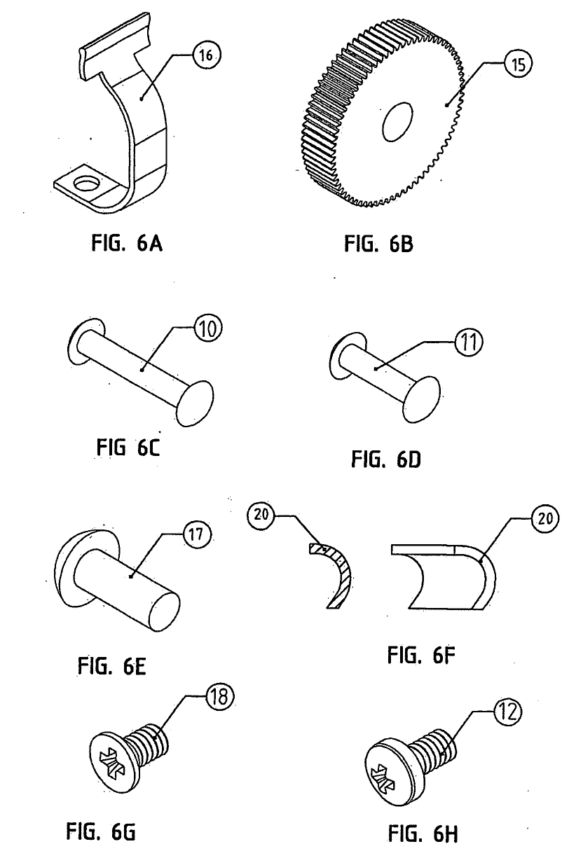

### DETAILED DESCRIPTION OF THE ILLUSTRATED EMBODIMENTS

Components of Automatic Maxillary Expander (7) and Transferring Apparatus (22)
1) Anterior column
1.1) Anti-rotation notches
2) Posterior column
2.1) Anti-rotation notches
3) Asymmetrical triangular prism-shaped Spike
3.1) Notches
4) Carrier block
5) Geared co-axial screw
5.1) Gear
5.2) Screw housing
5.3) Bores
6) Stability shaft
6.1) Shaft housings
7) Automatic Maxillary Expander
8) Fulcrum
9) Crowbar
10) Crowbar pin
11) Cradle gear pin
12) Retentive arc bolt
13) Cradle
14) Rack-and-pinion gear member
15) Gear
16) Retentive arc
17) Arc pin
18) Cradle bolts
19) Crowbar arc
20) Back stop
21) Handhold
22) Automatic Maxillary Expander Transferring Apparatus

Automatic Maxillary Expander (7) consists of two carrier blocks (4), a screw housing (5.2) formed in the middle part of the carrier blocks (4), shaft housings (6.1) formed on the carrier blocks (4), a geared co-axial screw (5), which is mounted in screw housing (5.2), stability shafts (6) which are situated on both sides of the geared co-axial screw (5) in shaft housings (6.1) and spikes (3) mounted on the anterior and posterior columns (1,2) which are fixed to carrier blocks (4) for providing penetration and anti-rotation of the automatic maxillary expander (7) into the palatal bone. The anterior column (1) has an anti-rotation notch (1.1) which prevents rotation into the palatal vault, the posterior column (2) has an anti-rotation notch (2.1) which prevents rotation into the palatal vault, and the spikes (3) have notches (3.1) formed on the wider surfaces thereof facing the tongue for providing stability and penetrating the spikes (3) deeply by cropping the palatal bone. The geared co-axial screw (5) includes two bores (5.3) formed thereon which are intersecting each other at 90 degrees in the middle. In addition, geared co-axial screw (5) includes a gear (5.1) having a certain number of teeth. Activation key is inserted in the opening of these bores (5.3) to rotate the geared co-axial screw (5). As the Automatic Maxillary Expander (7) engages the special Transferring Apparatus (22) with the help of the gear (5.1) on the geared co-axial screw (5), the practitioner can easily place it on the patient's palate.

The Practitioner inserts Automatic Maxillary Expander (7), which is attached to the special Transferring Apparatus (22), to the palate under local anesthesia and then triggers the crowbar (9). When the crowbar (9) is pressed, the part under the fulcrum (8) comes closed to handhold (21). Meanwhile, the crowbar arc (19) is suspended and the upper part of the crowbar (9) goes away from the handhold (21). This movement of the crowbar (9) draws away the rack-and-pinion gear member (14), connected to the upper part of the crowbar (9) from the cradle (13). Thus, the gear (15) in the cradle (13) moves counter-clockwise by the movement of the rack-and-pinion gear member (14). Therefore, geared co axial screw (5), planted between retentive arc (16) and backstop (20) over the cradle (13) tap, in the middle of Automatic Maxillary Expander (7) starts to revolve clockwise. So, Automatic Maxillary Expander (7) expands horizontally. Posterior columns (2) are longer than anterior columns (1), providing a better adaptation to palatal vault. The asymmetric triangular prism-shaped spikes (3) on the columns (1, 2) in both sides penetrate into palatal bone with each tour of the geared coaxial screw (5), then the carrier blocks (4) are separated from each other and the Automatic Maxillary Expander (7) is attached to the palate thanks to the spikes (3). The Automatic Maxillary Expander (7) placed in the palatal surface is stuck between the backstop (20) and flexible retentive arc (16). After this stage, Automatic Maxillary Expander (7) is removed from transferring apparatus (22) with the help of a gentle force and Automatic Maxillary Expander (7) remains stable in the palatal surface. Thus Automatic Maxillary Expander (7) accomplishes its task with the Transferring Apparatus (22).

Automatic Maxillary Expander Transferring Apparatus (22) enables the practitioner to place Automatic Maxillary Expander (7) into palatal surface with ease and precision. No current maxillary expander has such a transferring apparatus (22). This apparatus (22) enables the practitioner to place Automatic Maxillary Expander (7) in to the palate under local anesthesia in a very short time without any surgical operation. Thus, this Automatic Maxillary Expander (7), which is entirely different from the ones currently in use, has great advantage over them. In addition, this apparatus (22) is composed of a very simple mechanism. Automatic Maxillary Expander Transferring Apparatus (22) has been designed to help the practitioner to place Automatic Maxillary Expander (7) into palatal surface with ease and precision.

Automatic Maxillary Expander Transferring Apparatus (22) has an elliptic sectional shaped handhold (21), a fulcrum (8) fixed on to it, a crowbar (9) connected to fulcrum (8) with a crowbar pin (10). The crowbar arc (19) is positioned mutually between the bottom surface of the crowbar (9) and the upper surface of the handhold (21) with two arc pin (17). The cradle (13), which has a rack-and-pinion gear member (14), retentive arc (16) which holds the bottom of the automatic maxillary expander (7), gear (15) and back stop (20) which holds the upper part of the automatic maxillary expander (7) and which is fixed to the cradle (13) with solder, the cradle (13) is fixed on to the handhold (21) with two cradle bolts (18). The retentive arc (16) is connected to the cradle (13) with a retentive arc bolt (12), and the gear (15) is mounted into cradle (13) with the help of a cradle gear pin (11). The crowbar (9) and rack-and-pinion gear member (14) are connected to each other with a crowbar pin (10).

1. An automatic maxillary expander (7), being a bone-borne distractor for expanding the maxillary bone in adult and adolescents who have transversal maxillary hypoplasia, comprising:
two carrier blocks (4);
a screw housing (5.2) formed in the middle part of the carrier blocks (4);
shaft housings (6.1) formed on the carrier blocks (4);
a geared co-axial screw (5) which is mounted in screw housings (5.2);
bores (5.3) formed on the geared co-axial screw (5);
two stability shafts (6) which are situated on both sides of the geared co-axial screw (5) in shaft housings (6.1);
posterior columns (2) which are fixed to said carrier blocks (4) and having anti-rotation notches (2.1) which prevent rotation into the palatal vault;
anterior columns (1) which are fixed to said carrier blocks (4) and having anti-rotation notches (1.1) which prevent rotation into the palatal vault and formed shorter than the posterior notched columns (2) in order to provide better adaptation to the palatal vault; and
asymmetrical triangular prism-shaped spikes (3) which are mounted on the anterior and posterior columns (1,2) for providing penetration and anti-rotation of the automatic maxillary expander (7) into the palatal bone having notches (3.1) formed on the wider surfaces thereof facing the tongue for providing stability and penetrating the spikes (3) deeply by cropping the palatal bone.

2-11. (canceled)

12. The expander (7) according to claim 1, wherein the geared co-axial screw (5) comprises a gear (5.1) having a certain number of teeth in the middle.

13. An automatic maxillary expander transferring apparatus (22) used to place a automatic maxillary-expander (7) into the palate easily and appropriately, comprising:
a handhold (21) having elliptic-sectional shape;
a cradle (13) which is fixed on to the handhold (21);
a rack-and-pinion gear member (14) which is connected to the cradle (13);
a fulcrum (8) which is fixed on to the handhold (21);
a crowbar (9) which is connected to the fulcrum (8) and to the rack-and-pinion gear member (14) and which draws away the rack-and-pinion gear member (14) from the cradle (13) by each pressing movement thereof;
a crowbar arc (19) which is positioned mutually between the handhold (21) and the crowbar (9) and connected to both of them;
a flexible retentive arc (16) which is connected to the cradle (13) and holds the bottom of the automatic maxillary expander (7);
a back stop (20) which is fixed to the cradle (13) and holds the upper-part of the automatic maxillary expander (7) to fix it until it is planted in the palate; and
a gear (15) which is mounted into the cradle (13) and moves anticlockwise by the movement of the rack-and-pinion gear member (14) providing the expansion of the automatic maxillary expander (7) horizontally.

14. The transferring apparatus (22) according to claim 13, further comprising:
cradle bolts (18) which connect the cradle (13) to the handhold (21);
crowbar pins (10) which connect the crowbar (9) to the fulcrum (8) and to the rack-and-pinion gear member (14);
arc pins (17) which connect the crowbar arc (19) to the upper surface of the handhold (21) and to the bottom surface of the crowbar (9);
a retentive arc bolt (12) which connects the flexible retentive arc (16) to the cradle (13); and
a cradle gear pin (11) which provides mounting of the gear (15) into the cradle (13).
